(19) United States
(12) Patent Application Publication
(10) Pub. No.: US 2013/0144197 A1
(43) Pub. Date: Jun. 6, 2013

(54) ORTHOPEDIC DEVICE FOR DYNAMICALLY TREATING OSTEOARTHRITIS

(71) Applicant: Ossur hf, Reykjavik (IS)

(72) Inventors: Arni Thor INGIMUNDARSON, Gardabaer (IS); Adam DUNN, Irvine, CA (US); Jane LEE, Fullerton, CA (US); Harry Duane ROMO, Aliso Viejo, CA (US)

(73) Assignee: Ossur hf, Reykjavik (IS)

(21) Appl. No.: 13/693,416

(22) Filed: Dec. 4, 2012

Related U.S. Application Data
(60) Provisional application No. 61/567,176, filed on Dec. 6, 2011.

(51) Int. Cl. A61F 5/01

(52) U.S. Cl. CPC A61F 5/01
USPC 602/16

(57) ABSTRACT

An orthopedic device has upper and lower frames hingedly connected to each other by first and second primary hinges on opposed sides of the device and providing movement between flexion and extension. Each of the upper and lower frames includes opposed first and second medial-lateral portions connected by a central portion. A force strap is connected to the upper and lower frames and spiraling therebetween, and the force strap generally extends at a first location proximate to the first primary hinge and is generally urged in a first direction at the first location to the second primary hinge. The upper frame is rigid or semi-rigid, and includes means for permitting movement in proximal-distal directions and means for permitting movement in lateral-medial directions.

### FIELD OF ART

The present disclosure relates generally to the field of orthopedic and prosthetic devices, and more particularly to an orthopedic device that provides stability, protection, support, rehabilitation, and/or unloading to a portion of the human anatomy.

### BACKGROUND

Knee braces are widely used to treat a variety of knee infirmities. Such braces may be configured to impart forces or leverage on the limbs surrounding the knee joint to relieve compressive forces within a portion of the knee joint, or to reduce the load on that portion of the knee. In the event that knee ligaments are weak and infirm, a knee brace may stabilize, protect, support, unload, and/or rehabilitate the knee.

The knee is acknowledged as one of the weakest joints in the body, and serves as the articulating joint between the thigh and calf muscle groups. The knee is held together primarily by small but powerful ligaments. Knee instability arising out of cartilage damage, ligament strain and other causes is relatively commonplace since the knee joint is subjected to significant loads during the course of almost any kind of physical activity requiring using the legs.

A healthy knee has an even distribution of pressure in both the medial and lateral compartments of the knee. It is normal for a person with a healthy knee to place a varus moment on the knee when standing so the pressure between the medial and lateral compartments is uneven but still natural.

One type of knee infirmity that many individuals are prone to having is compartmental osteoarthritis. Compartmental osteoarthritis may arise when there is a persistent uneven distribution of pressure in one of the medial and lateral compartments of the knee. Compartmental osteoarthritis can be caused by injury, obesity, misalignment of the knee, or due to aging of the knee.

A major problem resulting from osteoarthritis of the knee is that the smooth cartilage lining the inside of the knee wears away. This leads to a narrowing of the joint space with developing cysts and erosions in the bone ends. Because of the narrowing of the joint, bone comes directly in contact with bone, and an uneven distribution of pressure develops across the knee which may cause the formation of bone spurs around the joint. All of these changes ultimately lead to increasing pain and stiffness of the joint.

While there are no cures to osteoarthritis, there are many treatments. Individuals who have a diagnosis of isolated medial or lateral compartmental osteoarthritis of the knee are confronted with a variety of treatment options such as medications, surgery, and nonsurgical interventions. Nonsurgical interventions include using canes, lateral shoe wedges, and knee bracing.

Knee bracing is useful to provide compartmental pain relief by reducing the load on the affected compartment through applying an opposing external valgus or varus moment about the knee joint. Unloading knee braces have been shown to significantly reduce osteoarthritis knee pain while improving knee function.

While known knee braces succeed at reducing pain or at stabilizing a knee joint, many users find these braces to be bulky, difficult to don, complicated to configure, and uncomfortable to wear. The embodiments described have streamlined features capable of providing relief for medial or lateral compartmental osteoarthritis, or functional stability of the knee while providing a configuration that has a low profile and unexpectedly provides a more conforming and supportive fit for the orthopedic device.

### SUMMARY

In an embodiment, the orthopedic device has upper and lower frames hingedly connected to each other by first and second primary hinges on opposed sides of the device and providing movement between flexion and extension. Each of the upper and lower frames includes opposed first and second medial-lateral portions connected by a central portion. A force strap is connected to the upper and lower frames and spiraling therebetween. The force strap extends at a first location proximate to the first primary hinge and is urged in a first direction at the first location to the second primary hinge. The upper frame is rigid or semi-rigid, and may include means for permitting movement in proximal-distal directions and may include means for permitting movement in medial-lateral directions. The upper and lower frame elements may be rigid or semi-rigid.

The orthopedic device may include an adjustment system coupling the force strap to the upper frame, and adjusted tension in the force strap. The adjustment system can have a regulation mechanism permitting incremental selective adjustment among a plurality of predetermined settings. The regulation mechanism may be a linear ratchet generally aligned along the length of the force strap and secured to an end portion of the force strap. Alternatively, the regulation mechanism may be a dial tensioning ratchet having a cable secured to an end portion of the force strap.

The orthopedic device may have a secondary hinge located between a first portion of the upper frame and the second primary hinge. The secondary hinge provides movement of the first portion relative to the lower frame along medial-lateral directions of the orthopedic device.

The second portion of the upper frame may have first and second segments movable relative to one another. The first and second segments movable upon tensioning of the force strap.

The orthopedic device may have a lock mechanism carried by one of the first and second segments and arranged to lock movement of the first and second segments relative to one another. The orthopedic device may also include a slide mechanism including a rack and pinion assembly permitting incremental movement of the first and second segments relative to one another. The slide mechanism may include a dial coupled to the rack for enabling adjustment of the slide mechanism.

According to a variation of the orthopedic device, the upper frame has an anterior side and has first and second medial-lateral side sections. The force strap extends and connects to the upper frame on the anterior side and on the first side medial-lateral, and spirals toward the second primary hinge within a periphery of the upper and lower frames, to secure to a posterior side of the lower frame element. The central portion of the upper frame may be on the anterior side of the device, and may be rigid or semi-rigid. The central portion of the upper frame may define a curved section spiraling between the first and second medial-lateral portions.

The orthopedic device disclosed may be of an unloading type knee brace, under the principles described in U.S. Pat. No. 7,198,610, granted Apr. 3, 2007, and U.S. Pat. No. 5,277,698, granted Jan. 11, 1994, both incorporated in their entirety by reference.

A method is provided for using the orthopedic device as a knee brace by tightening a force strap connected to the upper and lower frames by spiraling the strap between the upper and lower frames, with the first and second segments moving upon tensioning of the force strap such that at a first location the force strap generally extends proximate to the first primary hinge and is generally urged in a first direction at the first location to the second primary hinge. The knee brace and method may employ the features and steps described.

### BRIEF DESCRIPTION OF THE DRAWINGS

The orthopedic device is described referring to the accompanying drawings which show preferred embodiments according to the device described. The device as disclosed in the accompanying drawings is illustrated for example only. The elements and combinations of elements described below and illustrated in the drawings can be arranged and organized differently to result in embodiments still within the spirit and scope of the device described.

FIG. 1 is a front elevational view showing an embodiment of an orthopedic device in the form of a knee brace.

FIG. 2 is side elevational view of the knee brace according to FIG. 1.

FIG. 3 is a front elevational view showing another embodiment of a knee brace.

FIG. 4 is a rear elevational view showing the knee brace according to FIG. 3.

FIG. 5 is a front perspective view showing an embodiment of a slide mechanism in the knee brace of FIG. 1.

FIG. 6 is a rear perspective view showing an embodiment of the slide mechanism in FIG. 5.

FIG. 7 is a schematic view showing activation of the slide mechanism of FIG. 5 where the slide mechanism is in a neutral configuration.

FIG. 8 is a schematic view showing activation of the slide mechanism of FIG. 5 where the slide mechanism is a retracted configuration.

FIG. 9 is a schematic view showing activation of the slide mechanism of FIG. 5 where the slide mechanism is in an extended configuration.

FIG. 10 is front schematic view showing the slide mechanism and the lock mechanism in the knee brace of FIG. 1.

### DETAILED DESCRIPTION OF VARIOUS EMBODIMENTS

### A. Overview

A better understanding of different embodiments of the invention may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that, unless a term is expressly defined in this patent to possess a described meaning, there is no intent to limit the meaning of such term, either expressly or indirectly, beyond its plain or ordinary meaning

Any element in a claim that does not explicitly state “means for” performing a specified function, or “step for” performing a specific function, is not to be interpreted as a “means” or “step” clause as specified in 35 U.S.C. §112, paragraph 6.

### B. Definitions

For ease of understanding the disclosed embodiments of an orthopedic device, the anterior and posterior portions of the orthopedic device are described independently. The anterior and posterior portions of the orthopedic device function together to support and stabilize anatomical portions of the wearer of the device.

For further ease of understanding the embodiments of an orthopedic device as disclosed, a description of a few terms is necessary. As used, the term “proximal” has its ordinary meaning and refers to a location situated next to or near the point of attachment or origin or a central point, or located toward the center of the body. Likewise, the term “distal” has its ordinary meaning and refers to a location situated away from the point of attachment or origin or a central point, or located away from the center of the body. The term “posterior” also has its ordinary meaning and refers to a location behind or to the rear of another location. The term “anterior” has its ordinary meaning and refers to a location ahead of or to the front of another location.

The term “extension” is the movement or position of a joint is moving toward or in its most straight position. The term “flexion” is the movement or position of a bending or bent joint.

The terms “rigid,” “flexible,” “compliant,” and “resilient” may be used herein to distinguish characteristics of portions of certain features of the orthopedic device. The term “rigid” should denote that an element of the device is generally devoid of flexibility. Within the context of frame or support members or shells that are “rigid,” it is intended to indicate that they do not lose their overall shape when force is applied, and in fact they may break if bent with sufficient force. The term “flexible” should denote that features are capable of repeated bending such that the features may be bent into retained shapes or the features do not retain a general shape, but continuously deform when force is applied.

The term “compliant” is used to qualify such flexible features as conforming to the shape of another object when placed in contact therewith, via any suitable natural or applied forces, such as gravitational forces, or forces applied by external mechanisms, for example, strap mechanisms. The term “resilient” is used to qualify such flexible features as generally returning to an initial general shape without permanent deformation. As for the term “semi-rigid,” this term is used to connote properties of support members or shells that provide support and are free-standing; however such support members or shells may have some degree of flexibility or resiliency.

### C. Various Embodiments of the Orthopedic Device and Components for Use Therewith

The embodiment of the knee brace disclosed may be of an unloading, osteoarthritis knee brace of a type generally described in U.S. Pat. Nos. 7,198,610 and 5,277,698. The description focuses on the structure, materials, and configuration of a particular embodiment of an unloading, osteoarthritis knee brace, without belaboring the particular effects and modalities for treating osteoarthritis in the knee joint.

The illustrated knee brace 10 has a frame structure having a shape corresponding to the knee brace discussed in U.S. patent application Ser. No. 13/212,382, filed on Aug. 18, 2011. The actual shape of the knee brace is not essential, and such the shape of the knee brace in the illustrated embodiment is merely exemplary of one of many shapes that the knee brace may have under the invention.

The knee brace 10 includes upper and lower frames 11, 14 hingedly connected to each other by first and second primary hinges 16, 18 located on opposed sides of the device, particularly the medial and lateral sides. The primary hinges 16, 18 provide movement for the knee between flexion and extension.

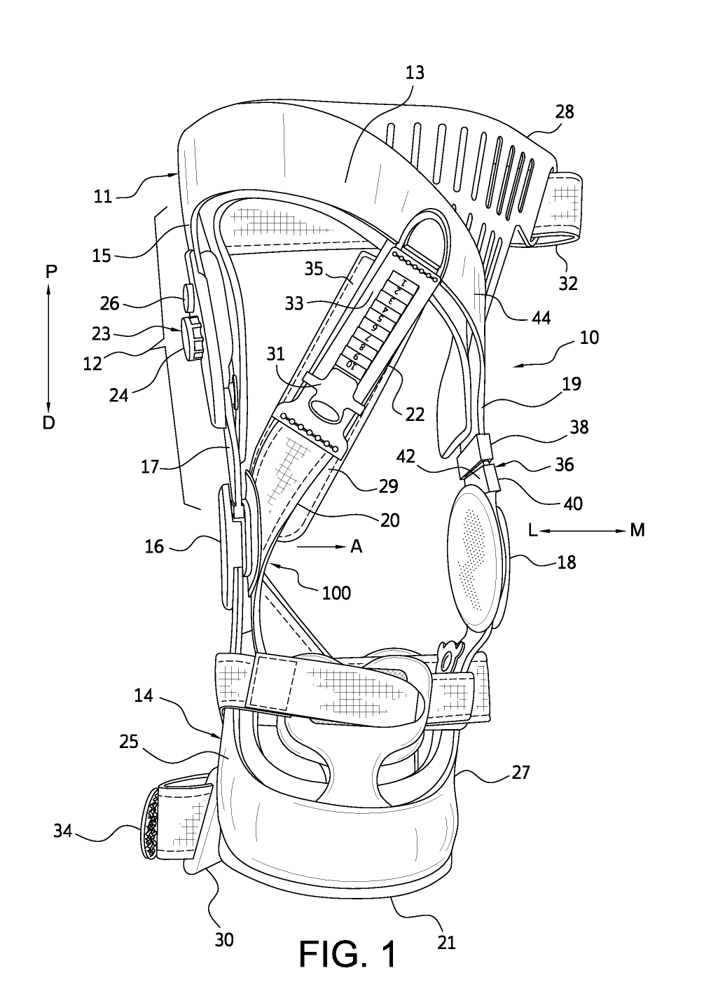

The upper frame 11 includes a first side portion 12 comprising first and second side segments 15, 17 preferably located on the lateral side of the brace and movable relative to one another. The first side portion 12 is secured to the first primary hinge 16. A second side portion 19 is on the medial side of the brace and connects to the second primary hinge 18 by a secondary hinge 36 providing movement in the lateral and medial directions (as generally depicted by line L-M in FIG. 1). An upper central portion 13 spirals downwardly from the first side portion 11 to the second side portion 19, and is on the anterior side of the brace.

The secondary hinge 36 includes a living hinge 42 at a middle portion of the hinge and bordered by top and bottom mounts 38, 40 which secure to the second side portion 19 and the second primary hinge 18, respectively. Various other hinge types may be employed including those taught in U.S. patent application Ser. No. 12/264,020, filed on Nov. 3, 2008.

A slide mechanism 23 is provided in order to control the movement between the first and second side segments 15, 17 generally in proximal and distal directions (as generally depicted by line P-D in FIG. 1). The slide mechanism 23 includes a dial 24 which permits incremental movement of the first and second segments relative to one another, and also includes a lock mechanism 26 that retains the first and second segments in positional relationship to one another.

The lower frame 14 includes first and second lower side portions 25, 27 generally on the lateral and medial sides of the brace. A lower central portion 21 connects to the first and second lower side portions 25, 27. The first and second lower side portions 25, 27 secure to the first and second primary hinges 16, 18, respectively.

Preferably, the upper and lower frames are rigid or semi-rigid. As an example, the frames may be formed from aluminum and may be malleable from a cold forming treatment so as to allow for easy customization of the frame to a particular leg shape. Other exemplary materials that may be used for constructing the frames include metals such as titanium, and steel, thermoset resin composite systems including glass or carbon fibers, and thermoplastics rendered rigid by way of material composition and geometry of the frames.

A flexible upper subshell 28 connects to the upper frame 11, and extends at least in an area not occupied by the upper frame 11 on the medial side of the brace. An upper circumferential strap 32 secures to the upper subshell 28 and extends about the posterior side of the brace and connects to the upper frame 11 and upper subshell 28 on the lateral side. Likewise, a flexible lower subshell 30 may connect to the lower frame 14. A lower circumferential strap 34 extends about the posterior side of the brace and connects to the flexible lower subshell 20 and the lower frame 14. One end of the lower strap 34 secures to the lower frame 14 by a buckle 46, that may be of the type described in U.S. Pat. No. 7,198,610.

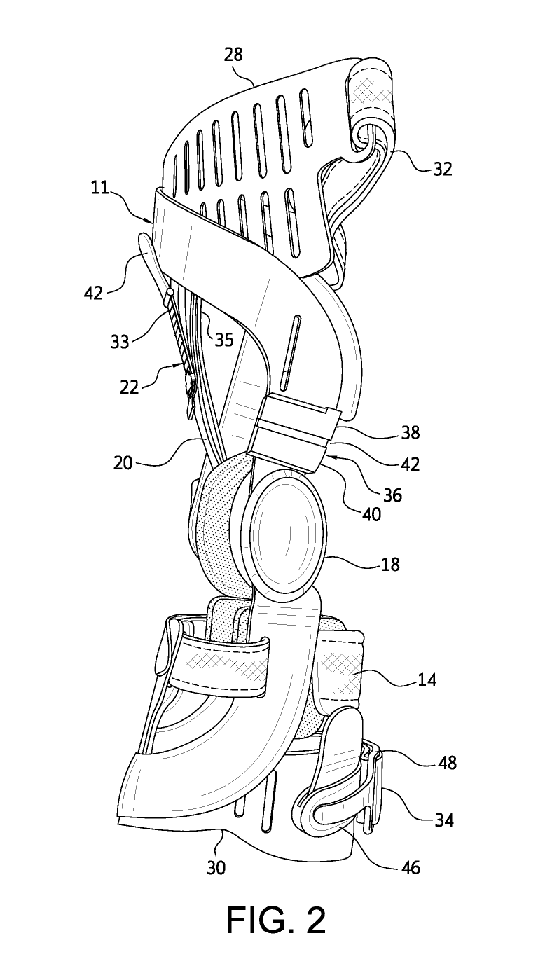

A force strap 20 connects to the upper frame 11 (at the anterior side) at the medial side of the central portion 13 and spirals downwardly toward the first primary hinge 16 and then down to the posterior side of either the lower frame 14 or the lower subshell 30. As shown in FIG. 2, the strap 20 secures to an angled section 48 of the buckle 46 to orient the strap 20 at an angle relative to the lower frame 14. The upper end 29 of the force strap 20 couples to the upper frame 11 by an adjustment system 22. The force strap 20 at or near a first location 100 the force strap generally extends proximate to the first primary hinge 16 and is generally urged in at least a first direction A at the first location 100 to the second primary hinge 18.

The depicted knee brace is defined as a double upright osteoarthritis (OA) brace due to there being side portions and hinges on both the lateral and medial sides. The strap 20 is confined within the periphery of the upper and lower frames, so it does not extend beyond any side of the upper and lower frames.

An embodiment of an orthopedic device is provided to reduce the effect of osteoarthritis in a knee joint, or stabilize a knee joint weakened by injury or other infirmities. Although the illustrated embodiment shows the secondary hinge positioned on the medial side of the orthopedic device and the force strap positioned to spiral along the lateral side of the orthopedic device, it will be understood that the orthopedic device may be configured to reduce or cure both medial and lateral knee joint infirmities, and the secondary hinge may be positioned on the lateral side of the orthopedic device and the force strap may be positioned to spiral along the medial side of the orthopedic device. Further, secondary hinges may also be positioned on both the lateral and medial sides of the orthopedic device.

As illustrated, the adjustment system 22 is similar to an adjustment system described in U.S. Pat. No. 7,198,610. The adjustment system 22 includes a ratchet slide 33 carried by the strap end 29, and engages a latch 31 carried by an attachment 35. The attachment 35 pivotally secures to the upper frame 11 and couples the strap 20 to the upper frame 11. The ratchet slide 33 is arranged to slide relative to the upper frame 11 at a plurality of predefined settings, and locks at one of the predefined settings due to the latch 31. A handle 44 provides for pulling the ratchet slide 33 toward the upper frame 11 to tension the strap 20.

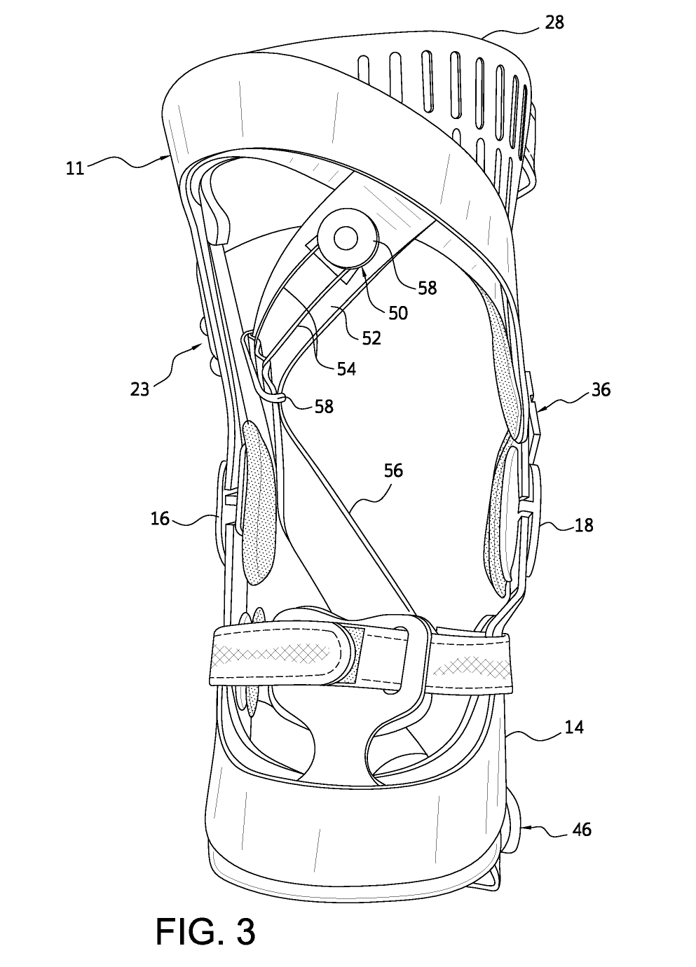

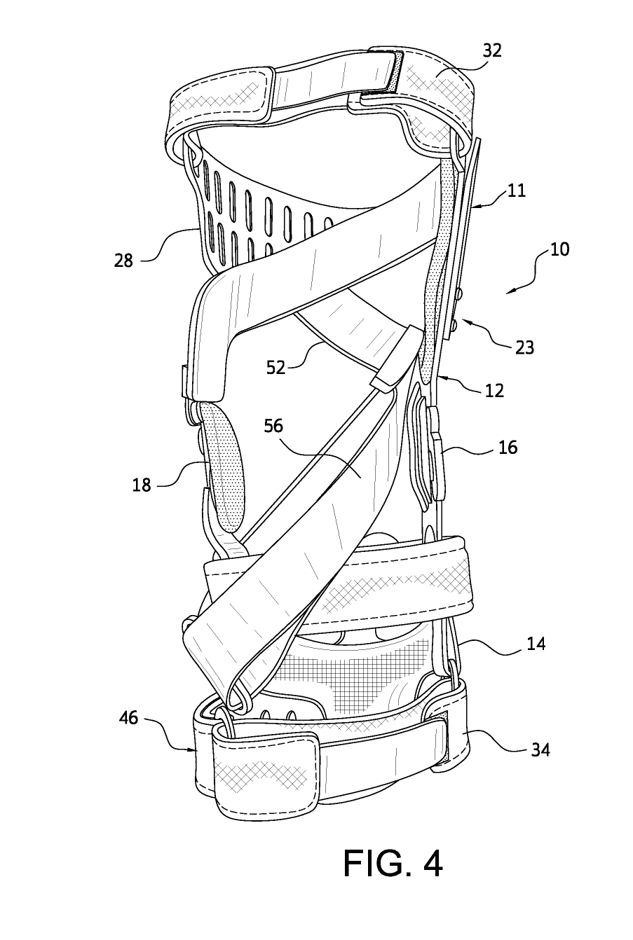

In a variation of an adjustment system, FIG. 3 shows a dial tensioner type adjustment system 50. According to this system, a strap mount 52 pivotally secures to the upper frame 11 at location similar as in FIG. 1. The strap mount 50 carries a dial tensioner 58 of a type described in U.S. Pat. No. 7,198,610, and connects to a strap 56 by a pair of cables 54. A guide 58 on the strap mount 52 is arranged to maintain position of the strap 56 relative to the strap mount 52. From this construction, as the dial tensioner 58 is rotated, the strap 56 is incrementally adjusted relative to the strap mount 52 to tension the strap 56 against the leg of a wearer of the knee brace.

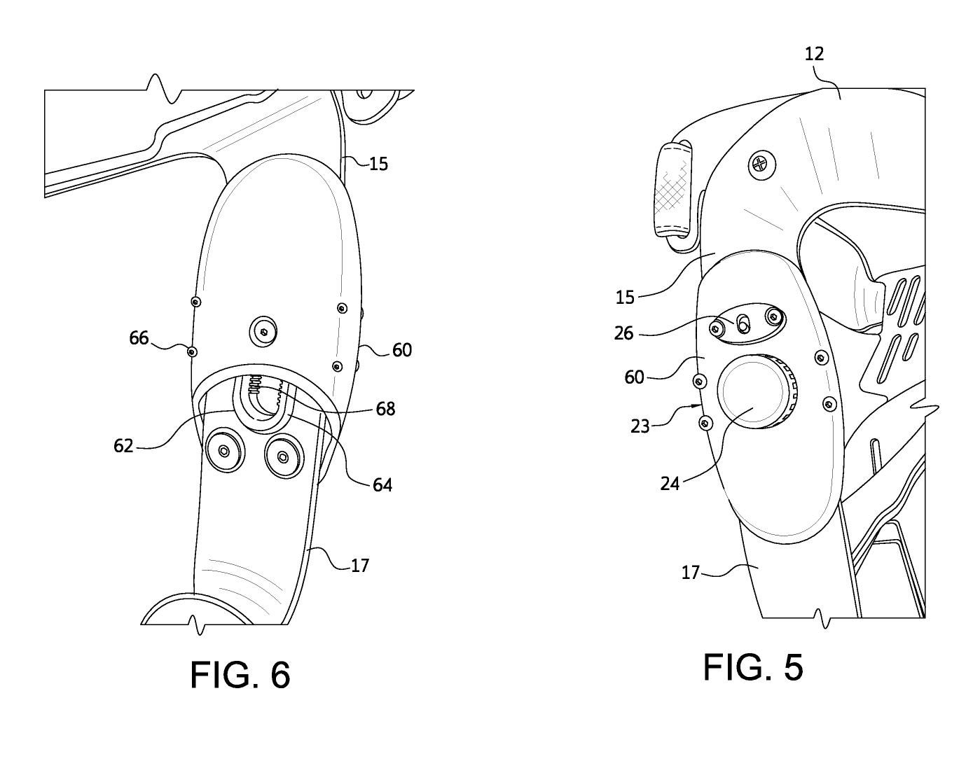

In reference to FIGS. 5-9, the slide mechanism 23 includes a casing 60 that contains the first and second segments 15, 17. In the example in FIG. 6, the second segment 17 includes an elongate slot 62 that receives an elongate rack insert 64 having a plurality of teeth 68. A rotatable pinion 70 is retained by a pin 66 secured to the casing 60. The pinion 70 carries a plurality of teeth arranged to engage the teeth 68 of the rack insert 64.

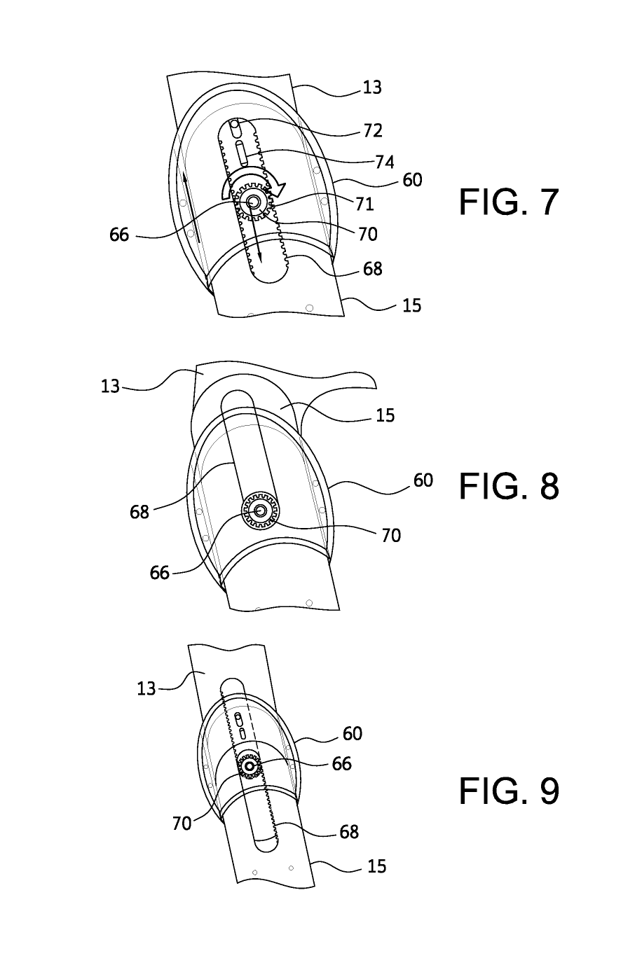

As shown in FIG. 7 when the first segment 13 is arranged in a middle or neutral position relative to the second segment 15, upon turning of the pinion 70 in a clockwise direction, the first segment 13 is brought downwardly relative to the second segment 15, shortening the length of the first upper side portion 11. FIG. 8 depicts the first and second segments 13, 15 as in the shortest or fully retracted configuration. Alternatively, FIG. 9 depicts the first and second segments 13, 15 as in the longest or fully extended configuration.

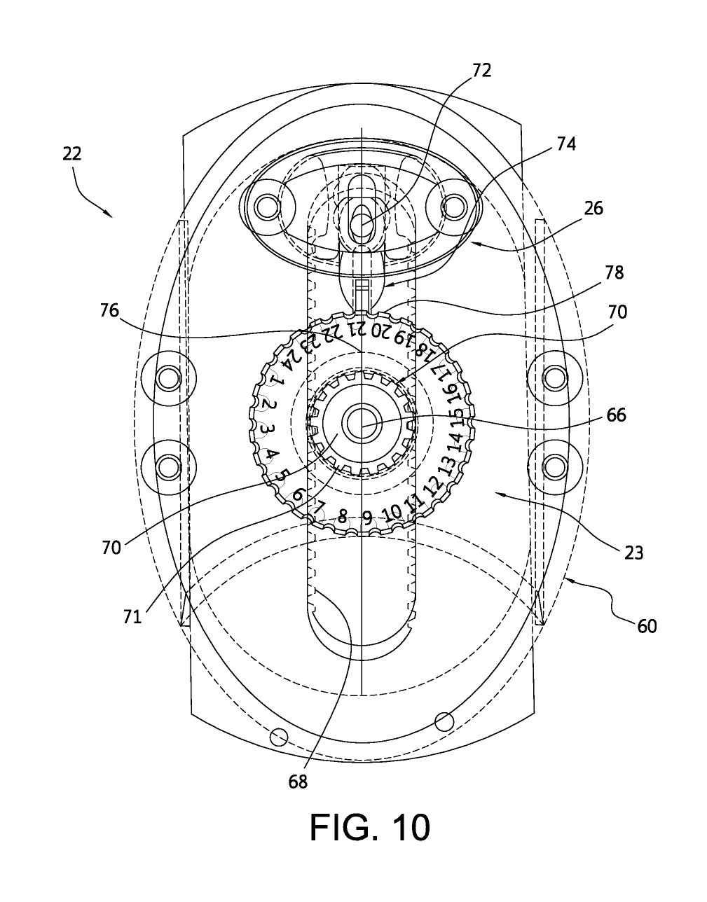

Turning to FIG. 10, the lock mechanism 26 is shown in more detail in that the lock mechanism 26 includes a pin or lever 72 that drops a block 74 having teeth into a second set of teeth 78 carried by a dial 76 used to turn the pinion 70. By dropping the block 74 into engagement with the dial 76, the pinion 70 is prevented from moving relative to the rack insert, hence maintaining the length of the first upper side portion 12.

The adjustment system, in part, allows for correction of leg curvature by forming a lateral inward or outward angle of the upper frame in relation to the lower frame, as discussed in U.S. Pat. No. 6,875,187, granted on Apr. 5, 2005, in relation to the securable slidable engager described.

The arrangement of the adjustment mechanism and the lock mechanism removes the need for the wearer of the brace to use any tool, which results in a brace easy to adjust. The rack and pinion arrangement of the slide mechanism allows the user to increase or decrease the alignment of the brace, and therefore create a force required to unload the knee and relieve pain.

1. An orthopedic device, comprising:
upper and lower frames hingedly connected to each other by first and second primary hinges located on opposed sides of the device and providing movement between flexion and extension, each of the upper and lower frames including opposed first and second medial-lateral portions connected by a central portion;
a force strap connected to the upper and lower frames and spiraling therebetween, wherein at a first location the force strap generally extends proximate to the first primary hinge and is generally urged in a first direction at said first location to the second primary hinge.

2. The orthopedic device according to claim 1, further comprising an adjustment system coupling the force strap to the upper frame, the adjustment system arranged to adjust tension in the force strap.

3. The orthopedic device according to claim 2, wherein the adjustment system has a regulation mechanism permitting incremental selective adjustment among a plurality of predetermined settings.

4. The orthopedic device according to claim 2, wherein the regulation mechanism is a linear ratchet generally aligned along the length of the force strap and secured to an end portion of the force strap.

5. The orthopedic device according to claim 2, wherein the regulation mechanism is a dial tensioning ratchet having a cable secured to an end portion of the force strap.

6. The orthopedic device according to claim 1, further comprising a secondary hinge located between a first portion of the upper frame and the second primary hinge, the secondary hinge providing movement of the first portion relative to the lower frame along medial-lateral directions of the orthopedic device.

7. The orthopedic device according to claim 1, comprising means for permitting movement in proximal-distal directions and means for permitting movement in medial-lateral directions.

8. The orthopedic device according to claim 1, wherein the upper and lower frame elements are rigid or semi-rigid.

9. The orthopedic device according to claim 1, wherein the second portion of the upper frame comprises first and second segments movable relative to one another, the first and second segments movable upon tensioning of the force strap.

10. The orthopedic device according to claim 9, further comprising a lock mechanism carried by one of the first and second segments and arranged to lock movement of the first and second segments relative to one another.

11. The orthopedic device according to claim 9, further comprising a slide mechanism including a rack and pinion assembly permitting incremental movement of the first and second segments relative to one another.

12. The orthopedic device according to claim 11, wherein the slide mechanism includes a dial coupled to the rack for enabling adjustment of the slide mechanism.

13. The orthopedic device according to claim 1, wherein the upper frame has an anterior side and has first and second medial-lateral side sections, the force strap extends and connects to the upper frame on the anterior side and on the first side medial-lateral, the force strap spirals toward the second primary hinge within a periphery of the upper and lower frames, and secures to a posterior side of the lower frame element.

14. The orthopedic device according to claim 13, wherein the central portion of the upper frame is located on the anterior side of the device, and is rigid or semi-rigid.

15. The orthopedic device according to claim 14, wherein the central portion of the upper frame defines a curved section spiraling between the first and second medial-lateral portions.

16. An orthopedic device, comprising:
upper and lower frames hingedly connected to each other by first and second primary hinges located on opposed sides of the device and providing movement between flexion and extension, each of the upper and lower frames including opposed first and second medial-lateral portions connected by a central portion, the second portion of the upper frame including first and second segments movable relative to one another in generally proximal-distal directions of the orthopedic device, whereas the first portion of the upper frame is a continuous segment without interruption; and
a force strap connected to the upper and lower frames and spiraling therebetween, the first and second segments movable upon tensioning of the force strap.

17. The orthopedic device according to claim 16, further comprising a secondary hinge located between a first portion of the upper frame and the second primary hinge, the secondary hinge providing movement of the first portion relative to the lower frame generally along a medial-lateral direction of the device.

18. The orthopedic device according to claim 16, wherein at a first location the force strap generally extends proximate to the first primary hinge and is generally urged in a first direction at said first location to the second primary hinge.

19. A method for using a knee brace having upper and lower frames hingedly connected to each other by first and second primary hinges located on opposed sides of the device and providing movement between flexion and extension, each of the upper and lower frames including opposed first and second medial-lateral portions connected by a central portion, the second portion of the upper frame comprising first and second segments movable relative to one another in a generally proximal-distal direction of the orthopedic device, the method comprising the steps of:
tightening a force strap connected to the upper and lower frames by spiraling the strap between the upper and lower frames, the first and second segments movable upon tensioning of the force strap such that at a first location the force strap generally extends proximate to the first primary hinge and is generally urged in a first direction at said first location to the second primary hinge.

20. The method according to claim 19, further comprising the step of adjusting the first and second segments of the second portion of the upper frame in generally proximal-distal directions upon extension and flexion of the knee brace.
